(19) United States
(12) Patent Application Publication
(10) Pub. No.: US 2007/0264199 A1
(43) Pub. Date: Nov. 15, 2007

(54) Magnetic nanoparticle composition and methods for using the same

(76) Inventors: Vinod D. Labhasetwar, Omaha, NE (US); Tapan K. Jain, Omaha, NE (US); Diandra Leslie-Pelecky, Lincoln, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/235,631

(22) Filed: Sep. 26, 2005

(51) Int. Cl. A61K 49/10; A61K 9/14

(52) U.S. Cl. 424/9.32; 424/489; 977/906

(57) ABSTRACT

The present invention is a magnetic nanoparticle composition with enhanced drug delivery characteristics. The magnetic nanoparticle composition is composed of a magnetic particle core surrounded by a fatty acid and surfactant corona. Methods for increasing the efficacy of therapeutic agents and facilitating diagnostic imaging are also provided.

### INTRODUCTION

Magnetic nanoparticles have emerged as effective drug delivery systems, as it is feasible to produce, characterize, and specifically tailor their functional properties for drug delivery applications (Gupta, et al. (2003) IEEE Trans. Nanobioscience 2:255-261; Gupta & Wells (2004) IEEE Trans. Nanobioscience 3:66-73; Zhang, et al. (2002) Biomaterials 23:1553-1561; Berry, et al. (2004) Int. J. Pharm. 269:211-225; Tiefenauer, et al. (1993) Bioconjug. Chem. 4:347-352; Alexiou, et al. (2000) Cancer Res. 60:6641-6648). An externally-localized magnetic-field gradient can be applied to a chosen site to attract drug-loaded magnetic nanoparticles from blood circulation (Alexiou, et al. (2002) J. Magn. Magn. Mater. 252:363-366). Drug targeting to tumors, or other pathological conditions, is desirable since therapeutic agents can demonstrate non-specific toxicities that significantly limit their therapeutic potential.

Magnetic nanoparticles generally are coated with hydrophilic polymers such as starch or dextran, and the therapeutic agent of interest is either chemically conjugated or ionically bound to the outer layer of polymer (Alexiou, et al. (2000) supra; Mehta, et al. (1997) Biotechnol. Tech. 11:493-496; Koneracka, et al. (1999) J. Magn. Magn. Mater. 201:427-430; Koneracka, et al. (2002) J. Mol. Catal. B: Enzym. 18:13-18; Bergemann, et al. (1999) J. Magn. Magn. Mater. 194:45-52). This approach is complex, involving multiple steps, and usually results in limited drug-loading capacity with the bound drug dissociating within hours (Alexiou, et al. (2000) supra). Rapid dissociation of drug from the carrier system reduces effectiveness, especially in cancer therapy where chronic drug retention in the target tissue is required for therapeutic efficacy. Entrapping magnetic nanoparticles into other sustained-release polymeric drug carrier systems such as in microparticles formulated from poly-dl-lactide-co-glycolide, polylactides, polyanhydrides (Chattopadhyay & Gupta (2002) Ind. Eng. Chem. Res. 41:6049-6058) or in dendrimers and other polymers, can result in significant loss in the magnetization (˜40 to 50%) of the core magnetic material (Strable, et al. (2001) Chem. Mater. 13:2201-2209; Ramirez & Landfester (2003) Macromol. Chem. Phys. 204:22-31). This decrease in magnetization negatively influences the magnetic targeting ability of the carrier system in vivo. The current approaches are further limited by the amount of magnetic nanoparticles that can be incorporated into drug delivery systems; for example, only 6% by weight α-Fe can be incorporated into silica nanospheres, which may not impart sufficient magnetic property to the formulation for effective targeting (Tartaj & Serna (2003) J. Am. Chem. Soc. 125:15754-15755). Ferrite particles encapsulated in polyglycidyl methacrylate have been disclosed which have 38 weight % of iron oxide (Nishibiraki, et al. (2005) J. Appl. Phys. 97:10Q919). Moreover, polystyrene nanoparticles with 39.1% magnetite loading have been reported (Ramirez & Landfester (2003) Macromol. Chem. Phys. 204:22-31), however, because polystyrene is not biodegradable, it is not compatible with use in humans. Further, the polystyrene entrapped magnetic nanoparticles has lower magnetization as compared to that of the original magnetic material.

Needed in the art is a magnetic particle with a high drug-loading capacity, a desirable release profile, high aqueous dispersion stability, biocompatibility with cells and tissue, and retention of magnetic properties after modification with polymers or chemical reaction. The present invention meets this long-felt need.

### SUMMARY OF THE INVENTION

The present invention is a magnetic nanoparticle composition composed of a magnetic particle core coated with a fatty acid and surfactant and a method for producing the same. In particular embodiments, the nanoparticle composition further contains a functional group, at least one therapeutic agent, or a detectable moiety.

Methods for increasing the efficacy of a therapeutic agent and facilitating imaging are also provided. In certain embodiments of the methods of the invention, the magnetic nanoparticle composition is delivered to a selected part of the body by exposing the selected part of the body to an external magnetic field.

### DETAILED DESCRIPTION OF THE INVENTION

A novel fatty acid- and surfactant-stabilized magnetic nanoparticle composition has now been developed. The instant composition is particularly desirable as it contains a single magnetic particle core per nanoparticle. Advantageously, hydrophobic compounds can be partitioned into the fatty acid corona surrounding the metal core and the surfactant, anchored at the interface of the fatty acid corona, confers an aqueous dispersity to the nanoparticle formulation. A water-dispersible nanoparticle formulation is achieved, without the loss of magnetic properties of the metal core.

By way of illustration an oleic acid-PLURONIC®-stabilized iron-oxide nanoparticle was prepared and loaded with doxorubicin (DOX). The hydrophilic nature of the iron-oxide nanoparticle surface precludes dispersal in non-polar solvents such as hexane and chloroform. Coating of iron-oxide nanoparticles with oleic acid hydrophobized the particle surface, thus the particles became dispersible in non-polar solvents. Complete coverage of iron-oxide nanoparticles with oleic acid was important to achieving uniform anchoring of PLURONIC® onto these particles for their dispersion in water. Increasing oleic acid concentration reduced particle sedimentation in hexane, as well as the mean particle size and polydispersity index. These data indicated that ˜23 weight % (of the total formulation content) or more oleic acid was required to disperse iron-oxide nanoparticles in hexane. To determine the amount of oleic acid that could be associated with the iron-oxide nanoparticles, formulations with different concentrations of oleic acid were characterized for mass loss using thermogravimetric analysis. The mass-loss data demonstrated an increase in bound oleic acid to iron-oxide nanoparticles with an increase in oleic acid concentration; however, no significant difference in the mass loss was observed when 17 or 23 weight % oleic acid was used, indicating a saturation binding of oleic acid to particle surface around these concentrations. The thermogravimetric analysis data demonstrated that ˜18 weight % oleic acid remained bound to nanoparticles when 23 weight % oleic acid was used in the formulation, i.e., 75 weight % of the added oleic acid was bound to the iron-oxide nanoparticles and could not be washed off. The particle-size-analysis data in hexane demonstrated that a higher amount of oleic acid (30 weight %) was required for dispersion of iron-oxide nanoparticles; however, the analysis demonstrated that ˜18 weight % oleic acid could be bound to nanoparticles. It is believed that this discrepancy in the amount of oleic acid required could be due to partial desorption of oleic acid from the nanoparticle surface when they were dispersed in hexane.

Thermogravimetric analysis and Fourier Transform Infrared (FT-IR) spectroscopy of oleic acid-coated iron-oxide nanoparticles indicated chemisorption of oleic acid at the iron-oxide nanoparticle surface and its multilayer deposition at higher than 17 weight % oleic acid concentration. The thermogravimetric analysis data demonstrated that the mass loss in oleic acid-coated nanoparticles occurred at about 300° C. (range 210-400° C.), which is higher than that for the pure oleic acid (250° C., range 150-400° C.). It is believed that this shift in the temperature could be due to chemisorption of oleic acid on the iron-oxide nanoparticle surface, requiring higher temperature for the vaporization of bound oleic acid. The peak observed at 1705 cm⁻¹ in the FT-IR spectra of pure oleic acid was due to the C═O stretch dimer H-bonded, the broad peak observed at around 3000 cm⁻¹ was due to the O—H stretch dimer H-bonded, and the peaks at 2854 cm⁻¹ and 2922 cm⁻¹ corresponded to the symmetric and asymmetric CH₂ stretching modes, respectively. The spectra of oleic acid-coated iron-oxide nanoparticles, however, lacked the C═O stretch at 1705 cm⁻¹, indicating binding of the carboxylic group of oleic acid to the iron-oxide nanoparticles. The spectra of pure iron-oxide and oleic acid-coated iron-oxide nanoparticles, showed that both stretching modes appeared in the spectrum: the symmetric stretching band was located at 1435 cm⁻¹ and the asymmetric band ranged from 1530 cm⁻¹ to 1570 cm⁻¹. The additional feature that appeared at 1712 cm⁻¹ could have been due to the C═O stretch monomer. This peak started to appear for concentrations of oleic acid higher than 17 weight %, and could be evidence of oleic acid bilayer formation. A strong and broad peak at 3454 cm⁻¹ indicated chemisorption of oleic acid onto iron-oxide nanoparticles; however, the intensity of this peak decreased with increasing oleic acid concentration. The suppression of the OH vibrational mode in the 3000-3700 cm⁻¹ region has been related to evidence of host-guest interaction as a consequence of water release upon chemisorption of oleic acid. The ratio of the intensities of the CH₂ symmetric stretch mode to the OH stretch mode versus the relative concentration of oleic acid to iron-oxide showed a nearly constant value when the oleic acid concentration was about 17 weight %, indicating that oleic acid had reacted with most of the active binding sites on the iron-oxide nanoparticle surfaces. Using the average particle diameter of 9.3 nm for iron-oxide nanoparticles, at 17 weight % oleic acid concentration, the surface area occupied per oleic acid molecule was estimated to be 0.34 nm²; whereas, at 30 weight % oleic acid concentration, it was 0.21 nm². This decrease in surface area per oleic acid molecule at higher concentration of oleic acid indicates the formation of a multilayer coating. The thermogravimetric analysis of oleic acid-coated iron-oxide nanoparticles also demonstrated multilayer deposition of oleic acid at higher concentrations. Based on these observations, the formulation containing 23 weight % oleic acid with respect to total formulation weight, which is slightly in excess of that required for monolayer adsorption of oleic acid, was used for further studies.

The amount of PLURONIC® required to disperse oleic acid-coated iron-oxide nanoparticles in water also was determined. Increasing the PLURONIC® concentration up to 100 mg (19 weight % with respect to total formulation weight) reduced the particle size, but further increasing PLURONIC® concentration had an insignificant effect on particle size when measured by dynamic laser light scattering technique. The mass loss from thermogravimetric analysis indicated that 71 weight % of the added PLURONIC® was associated with nanoparticles when 100 mg PLURONIC® was added in the formulation. Lack of change in the particle size with increasing amounts of PLURONIC® may have been due to saturation of the oleic acid-water interface with PLURONIC®, thus the increase in PLURONIC® concentration beyond 100 mg had no further influence on the dispersibility of particles in water. The mean hydrodynamic particle size measured by dynamic laser light scattering analysis was 193 nm with a polydispersity index of 0.262, whereas the particle size calculated by analyzing the X-ray diffraction peaks using the integral-breath method was 9.2±0.8 nm and that from transmission electron microscopy (TEM) was 11±2 nm. The larger particle size by laser light scattering, which measures the hydrodynamic diameter, could be due in part to the contribution of oleic acid and PLURONIC® associated with nanoparticles, and hydration of the particle with water. The high polydispersity index also indicates that there is some aggregation of oleic acid-PLURONIC® stabilized nanoparticles when dispersed in water. This aggregation could be the result of incomplete dispersion of oleic acid-coated nanoparticles in PLURONIC® or due to their flocculation because these nanoparticles have almost neutral zeta potential (ζ=−0.22 mV). The zeta potential of uncoated iron-oxide nanoparticles was −13.40 mV, which could have been masked by the bound oleic acid and the coating of nonionic PLURONIC®. Since the concentration of PLURONIC® used in the formulation was below the critical micelle concentration (cmc=20 mg/mL; Desai, et al. (2001) Colloid Surf., A 178:57-69), it is possible that PLURONIC® could have been anchored at the interface of oleic acid-coated nanoparticles in the form of a multilayer deposit rather than as micelles.

The FT-IR spectra of oleic acid-PLURONIC®-stabilized iron-oxide nanoparticles at different concentrations of oleic acid and PLURONIC® demonstrated that there was no bonding of PLURONIC® to the particle surface in the absence of oleic acid. This was evident from the identical spectra of PLURONIC®-iron-oxide nanoparticles and pure iron-oxide nanoparticles; however, PLURONIC® bonding to nanoparticles increased with increasing oleic acid concentration. The FT-IR spectra of oleic acid-PLURONIC®-stabilized iron-oxide nanoparticles demonstrated broad bands around 1250 cm⁻¹-1000 cm⁻¹ that were due to the CH₂ rocking and C—O—C stretch vibrations of PLURONIC®. The FT-IR spectrum developed strong and well-defined bands at around 1113 cm⁻¹, typical of a block copolymer in the optimal formulation in which oleic acid completely covers the iron-oxide nanoparticle surface. The peaks at 2854 cm⁻¹ and 2920 cm⁻¹ in the spectra were due to chemisorbed oleic acid.

The optimized iron-oxide nanoparticle formulation was composed of 70.1 wt % iron-oxide, 15.4 weight % oleic acid and 14.5 weight % PLURONIC® (nominal composition was 63.0 weight % iron-oxide, 18.3 weight % oleic acid and 18.7 weight % PLURONIC®). The composition was determined based on the mass-loss data from the thermogravimetric analysis of oleic acid-coated and oleic acid-PLURONIC®-stabilized formulations. The iron content in this formulation was higher than that in a starch-coated iron-oxide formulation used in tumor drug delivery (50.8% vs. ˜1%; Alexiou, et al. (2000) supra). The X-ray diffraction spectra of oleic acid-PLURONIC®-stabilized iron-oxide nanoparticles exhibited peaks that corresponded to both maghemite (Fe₂O₃) and magnetite (Fe₃O₄).

The saturation magnetization M_{S}, coercivity H_{c} (at 10 K) and the peak temperature of the zero-field-cooled (ZFC) magnetization of oleic acid-PLURONIC®-stabilized iron-oxide nanoparticles are presented in Table 1.

TABLE 1

| | | | Coercive |
|---|---|---|---|
| | Saturation | | Field |
| | Magnetization | Tₘₐₓ | H_{C}(Oe) |
| Samples | M_{S} (emu/g) | (K) | at 10 K |
| Iron-oxide | 66.1 ± 0.1 | 215 ± 7 | 201 ± 11 |
| nanoparticles | | | |
| Oleic acid-PLURONIC ®- | 86.1 ± 0.5 | 170 ± 5 | 158 ± 05 |
| stabilized iron-oxide | | | |
| nanoparticles | | | |
| Drug loaded oleic acid- | 88.8 ± 0.5 | 160 ± 5 | 151 ± 06 |
| PLURONIC ®-stabilized | | | |
| iron-oxide | | | |
| nanoparticles | | | |

The M_{S} values were normalized assuming 100% magnetite for simplicity using the iron mass as determined by atomic absorption spectroscopy (Pepic, et al. (2004) Int. J. Pharm. 272:57-64). Hysteresis loops indicated negligible coercivity at room temperature, and the magnetization at 1.2 T (after subtracting a diamagnetic background) was 59.2±0.8 emu/g_{magnetite} for oleic acid-PLURONIC®-stabilized iron-oxide nanoparticles and 45.1±0.8 emu/g_{magnetite} for uncoated iron-oxide nanoparticles. The hysteresis loops measured at 300 K were fit to a Langevin function weighted by a log-normal distribution of particle sizes to determine the magnetic volume of the nanoparticle. The mean magnetic diameter was 9.9 nm±5.5 nm (mean±standard deviation). The nanoparticles were ferromagnetic at 10 K. The saturation magnetization at 10 K for oleic acid-PLURONIC®-stabilized iron-oxide nanoparticles was higher than that of unmodified iron-oxide nanoparticles and hysteresis developed. Table 1 shows the ZFC peak position (Tₘₐₓ) for the uncoated iron-oxide nanoparticles and for the optimized nanoparticle formulations. The peak temperature was determined from the derivative of the magnetization versus temperature. A higher temperature is indicative of interparticle interactions, as the magnetic nanoparticle size was constant.

DOX loading in formulation was 8.2±0.5 weight % (i.e., 82 μg drug per mg nanoparticles) with an encapsulation efficiency of 82% (i.e., 82% of the added drug was entrapped in the formulation). Since a magnetic field was used to separate drug-loaded magnetic nanoparticles, any drug that did not partition in the oleic acid corona surrounding the nanoparticles was retained in the aqueous phase. Drug loading did not change the magnetic properties of the formulation (Table 1). The release of DOX from nanoparticles was sustained, with about 28% cumulative drug release occurring in two days and about 62% over one week.

Control nanoparticles without drug did not show a cytotoxic effect in the concentration range of 0.1 to 100 μg/mL, as the cell growth rate with nanoparticles was the same as that of the medium control. The data thus indicate that surface modification with oleic acid and PLURONIC® does not cause a toxic effect. Drug-loaded nanoparticles, however, demonstrated a dose-dependent cytotoxic effect both in MCF-7 and PC3 cells, which was slightly lower than that observed with equivalent doses of the drug in solution. This could be because of the sustained drug-release property of the nanoparticles, as only about 40% of the loaded drug was released (based on the in vitro release data) during the experimental period of five days. Since the medium and control nanoparticles without drug demonstrated similar growth curves, the antiproliferative effect seen with drug-loaded nanoparticles was because of the drug effect.

Confocal laser scanning microscopy indicated internalization of DOX-loaded nanoparticles in MCF-7 cells within 2 hours of incubation. Drug was seen localized in the cytoplasm, indicating that it was associated with nanoparticles. Similar experiments with drug in solution demonstrated nuclear localization of the drug. Since drug-loaded nanoparticles demonstrated cytotoxic effect, the drug was released slowly from the nanoparticles in the cytoplasm, and then diffused into the nucleus, the site of action. Confocal microscopy of cells treated with drug-loaded nanoparticles for 24 and 48 hours showed that the drug was localized in the nucleus. Further, the fluorescence intensity in the nucleus was reduced slowly with incubation time in cells treated with drug in solution, whereas it increased in cells treated with drug-loaded nanoparticles. Accordingly, drug-loaded nanoparticles act as an intracellular depot and sustain drug retention.

Loading of a combination of different anticancer agents into a single magnetic nanoparticle formulation was also demonstrated. Paclitaxel and doxorubicin were selected for this analysis because paclitaxel acts via inhibiting mitosis by binding to microtubules, thus preventing cell mitosis, whereas doxorubicin acts by intercalating with the nuclear DNA and thus affecting many functions of DNA including DNA and RNA synthesis, thereby leading to cell apoptosis. The results demonstrated that a combination of drugs could be incorporated in magnetic nanoparticles with over 80% efficiency; one drug does not affect the loading efficiency of the other drug (Table 2).

TABLE 2

| | | | | Total Drug |
|---|---|---|---|---|
| Doxorubicin | | Paclitaxel | | Loading |
| Added | Loaded | Added | Loaded | (Mean ± SEM) |
| (% w/w) | (% w/w) | (% w/w) | (% w/w) | (% w/w)* |
| 0.0 | 0.0 | 10.0 | 9.5 | 9.5 |
| 5.0 | 3.7 | 5.0 | 4.8 | 8.5 |
| 10.0 | 8.2 | 0.0 | 0.0 | 8.2 |

*n = 2 or 3.

Although the IC₅₀ values for paclitaxel and the combination of drugs (1:1 paclitaxel and doxorubicin) either in solution or loaded in magnetic nanoparticles were nearly the same, the dose of paclitaxel used in the combination was half of that used alone (Table 3). Thus, by combining paclitaxel with doxorubicin, the amount of paclitaxel required for the same IC₅₀ was 50%. The dose of doxorubicin used in the combination, if used alone, was not effective. Thus, paclitaxel in combination with doxorubicin achieves the same antiproliferative effect but at a lower dose.

TABLE 3

IC₅₀ (ng/mL ± SEM)*

| Anticancer Agent | Soluble | Nanoparticle |
|---|---|---|
| Paclitaxel | 9.8 ± 0.5 | 10.6 ± 0.6 |
| Doxorubicin | 102.9 ± 17.8 | 795.5 ± 177 |
| Paclitaxel + Doxorubicin | 3.4 ± 2.05 | 15.5 ± 2.7 |

*n = 6.

The effects of iron-oxide nanoparticles on liver toxicity following intravenous administration were also assessed. Results of this analysis indicated that a slight surge in the serum aspartate aminotransferase (AST) level was apparent at 24 hours after injection of magnetic nanoparticles, but the level returned within the normal range thereafter (Table 4). However, alanine aminotransferase (ALT), alkaline phosphatase (AKP), and gamma-glutamyl transferase (GGT) enzyme levels were in the normal range. The transient increase in AST level may have been the result of response of the liver to particulate injection.

TABLE 4

| Time (Day) | AST (IU/L) | ALT (IU/L) | AKP (IU/L) | GGT (IU/L) |
|---|---|---|---|---|
| 0 | 139 ± 38 | 86 ± 1 | 166 ± 21 | 15 ± 0 |
| 0.25 | 193 ± 67 | 97 ± 1 | 130 ± 12 | 15 ± 0 |
| 1.0 | 385 ± 26 | 148 ± 10 | 182 ± 10 | 25 ± 0 |
| 7.0 | 168 ± 48 | 86 ± 1 | 116 ± 8 | 15 ± 0 |
| 14.0 | 113 ± 32 | 211 ± 1 | 201 ± 1 | 30 ± 0 |
| 21.0 | 93 ± 30 | 58 ± 4 | 128 ± 1 | 6 ± 1 |

Iron content in the serum collected at 21 days was also in the normal range (Table 5), indicating that iron has been cleared from the body. Since transferrin is synthesized in the liver, it is also used as an indicator of the liver function. Iron binding capacity, which reflects transferrin content, was in the normal range.

TABLE 5

| | Assay | | |
|---|---|---|---|
| | Control | Rat #1 | Rat #2 |
| Iron Level (μg/dL) | 123 | 118 | 155 |
| Iron Binding Capacity (μg/dL) | 528 | 504 | 570 |
| % Iron Saturation | 23 | 23 | 27 |

Histological analysis of the liver from animals injected with nanoparticles was similar to that of the control animal. Liver sections did not show any untoward change in the morphology of either heptocytes or Kupffer cells. Iron-oxide nanoparticles in Kupffer cells, which appear as a black deposit, were not observed, thus further indicating that iron had been cleared from the body. Moreover, there was no change in the behavior of the animals following nanoparticle injection. The overall data thus indicate normal liver function and no toxic effect of the instant magnetic nanoparticles.

Uptake of the instant nanoparticle formulation in ischemic and normal brain tissue in a rat cerebral ischemia model was analyzed in the presence of an external magnetic field. Infarcted rat brain, with no magnetic nanoparticles and no magnetic field served as a control. MRI scans of a control rat showed no oleic acid-PLURONIC®-stabilized iron-oxide nanoparticles in the brain. Several nanoparticles were found in the ischemic portion of rat brains injected with magnetic nanoparticles without magnetic field. From the complete MRI scan, it was possible to map the damaged area of the brain by tracing the distribution of nanoparticles. When nanoparticles were injected into a rat that was subjected to a magnetic field, the overall MRI scan was darker with intense dark spots in ischemic regions, indicating a greater accumulation of magnetic nanoparticles in the damaged regions of the brain in response to the external magnetic field.

The instant nanoparticle composition was further modified to incorporate a functional group on the surface of the coated particles for conjugation of targeting moieties such as antibodies and the like. The functional group was a carboxyl group provided by polyethylene glycol (PEG). When PEG and PLURONIC® were combined and coated onto iron-oxide nanoparticles, the dispersion of the iron-oxide nanoparticles was significantly improved when compared to either compound used alone. The average number of PEG molecules conjugated to iron-oxide nanoparticles was calculated indirectly by measuring the amount of PEG that was not conjugated to nanoparticles. For this purpose, FITC-conjugated PEG was employed and the washings were collected to determine the amount of FITC-PEG that did not bind to the nanoparticles. The average number of PEG molecules conjugated per magnetic nanoparticle (for 1:10 PEG:nanoparticle ratio) was calculated by dividing the number of PEG molecules bound to nanoparticles by the calculated average number (n) of nanoparticles using the equation n=6m/(Π×D³×ρ), wherein m is the nanoparticle weight, D is the number based on mean nanoparticle diameter determined by TEM, and ρ is the nanoparticle weight per volume unit (density), estimated to be 5.16 g/cm³. The amount of PEG conjugated was 82 μg/mg magnetic nanoparticles, which represents approximately 42 PEG molecules per nanoparticle.

Following MRI scanning, each brain was sectioned into 2 mm thick slices. The brain sections from the animal in which the magnetic field was applied appeared darker than the brain sections from the other animals. These sections were analyzed for magnetic properties and relative intensity of magnetic nanoparticles in different areas of the brain. Tissue collected from the ischemic area demonstrated higher magnetization (using SQUID) than that collected from the nonischemic area, indicating greater localization of magnetic nanoparticles in the ischemic area of the brain. Quantitative analysis of the magnetic nanoparticle levels with and without magnetic field indicated that uptake in brain with the magnetic field was three-fold higher than without the magnetic field (1.49 μg/g vs. 0.5 μg/g wet tissue, respectively). The SQUID analysis of brain sections thus compliments the MRI analysis of the brain for relative distribution of magnetic nanoparticles in ischemic verses nonischemic parts of the brain.

The circulation time of oleic acid-PLURONIC®-stabilized iron-oxide nanoparticles was monitored in rats following intravenous administration. Oleic acid-PLURONIC® stabilized nanoparticles (1.3 mg) were loaded with fluorescent dye (6-coumarin) and injected into rats. Blood was withdrawn from the tail vein at different time points and subsequently analyzed for nanoparticle levels. The prolonged retention of PLURONIC® coated nanoparticles in the blood indicated that the coating enhanced the circulation time of the nanoparticles. These results also indicate that PLURONIC® remained associated with the nanoparticles following systemic administration. Typically, nanoparticles that are not coated with hydrophilic polymers such as PLURONIC® or PEG disappear rapidly from the blood circulation following their systemic administration (Vandorpe et al. (1997) Biomaterials 18:1147-52).

The instant magnetic nanoparticle composition, also referred to herein as a formulation, offers several advantages over known magnetic nanoparticle formulations. The fatty acid corona layer allows for hydrophobic drug partitioning, a process much simpler than chemical conjugation of drugs, and provides a greater degree of flexibility in terms of loading of different water-insoluble drugs either alone or in combination. Further, the instant coating does not significantly affect magnetization. Moreover, the surfactant coating provides increased circulation time in vivo.

Accordingly, the instant invention is a nanoparticle composition composed of a magnetic particle core coated with a fatty acid and surfactant. As used herein, the terms coated or coating are used to refer to the process of adsorption (e.g., chemisorption or physical adsorption) of the fatty acid to the magnetic particle core and further van der Waals and non-polar group interactions between the surfactant and fatty acid. As such, the fatty acid and surfactant form an amphiphilic corona around the magnetic particle core thereby facilitating incorporation of hydrophobic moieties into the nanoparticle composition. In particular embodiments, a single (i.e., one) magnetic particle core is associated with each individual nanoparticle. As such, the concentration of components of the instant nanoparticle composition is uniform. The magnetic particle core is generally composed of a magnetic or magnetically responsive particle that is small enough in size to diffuse into tissues and enter cells (by endocytotic processes), yet large enough to respond to an applied magnetic field at 37° C. Thus, particles less than 100 nm in diameter, or desirably in the range of 1 to 50 nm are suitable for use in the present invention, wherein particle size can be dependent upon the material used for fabricating the instant particle.

The material forming the core can be any metal or combination of metals including iron, cobalt, zinc, cadmium, nickel, gadolinium, chromium, copper, manganese, and their oxides. The magnetic particle can also be composed of an alloy with a metal such as gold, silver, platinum, or copper. The invention further provides that the magnetic particle can be composed of a free metal ion, a metal oxide, a chelate, or an insoluble metal compound. In certain embodiments, the magnetic particle is fabricated from Fe₃O₄, Fe₂O₄, FeₓN, FeₓPt_{y}, CoₓPt_{y}, MnFeₓO_{y}, CoFeₓO_{y}, NiFeₓO_{y}, CuFeₓO_{y}, ZnFeₓO_{y}, and CdFeₓO_{y}, wherein x and y vary depending on the method of synthesis. In other embodiments, the magnetic particle is further covered with a layer of silicon; polymer; or a metal including gold, silver, iron, cobalt, zinc, cadmium, nickel, gadolinium, chromium, copper, and manganese, or an alloy thereof. In particular embodiments, the magnetic nanoparticle is a monocrystalline iron oxide nanoparticle (MION), e.g., as described in U.S. Pat. No. 5,492,814, U.S. Pat. No. 4,554,088, U.S. Pat. No. 4,452,773; U.S. Pat. No. 4,827,945, and Toselson, et al. (1999) Bioconj. Chemistry 10:186-191; chelate of gadolinium; superparamagnetic iron oxide particles (SPIOs); ultra small superparamagnetic iron oxide particles (USPIOs); or cross-linked iron oxide (CLIO) particles (see, e.g., U.S. Pat. No. 5,262,176). FeₓN, wherein x is 2 to 4, is particularly useful because of the variety of different magnetic properties which can be achieved. A giant moment Fe₁₆N₂ phase with Mₛ from 240-315 emu/g has been described. Further, Fe₄N has an Mₛ of 186-188 emu/g and Fe₃N has Mₛ values ranging from 43-160 emu/g (Nakatani & Furubayashi (1990) J. Magn. Magn. Mater. 85:11-13; Yamaguchi, et al. (2000) J. Magn. Magn. Mater. 215:529-531). X-ray and electron diffraction indicate that pure and multi-phase nanoparticles of FeₓN (x=2, 3, and 4) can be produced. Fe₃N can have coercivity up to 1000 Oe, and can be used for simultaneous drug delivery and hyperthermia applications. Moreover, FeₓN nanoparticles are acid-resistant making them useful for applications in acidic environments. Advantageously, Fe₄N can be significantly more oxidation resistant than pure Fe and have higher magnetization than iron oxides (Mₛ=70-100 emu/g for iron oxide). Cobalt-based nanoparticles are also contemplated due to their higher saturation magnetizations (i.e., Mₛ for Fe₅₀Co₅₀ alloy is 243 emu/g). In certain embodiments, the instant nanoparticle composition has a saturation magnetization of at least 50 emu/g. In other embodiments, the saturation magnetization of the instant nanoparticle composition is in the range of 80 to 300 emu/g.

Methods for producing magnetic particles are disclosed herein and generally well-known in the art. For example, to prepare magnetic particles with higher saturation magnetizations Mₛ, inert-gas condensation of fluids (IGC-F) was employed. Iron-based nanoparticles fabricated with IGC-F displayed a mean size of 11.6 nm and a standard deviation of 2.2 nm, whereas cobalt-based nanoparticles displayed a mean size of 42 nm. Both the iron-based and cobalt-based nanoparticles exhibited a ferromagnetic behavior, which was retained at room temperature.

A fatty acid employed in the instant nanoparticle is a single chain of alkyl groups containing from 8 to 22 carbon atoms with a terminal carboxyl group (—COOH) and high affinity adsorption (e.g., chemisorption or physical adsorption) to the surface of the magnetic particle. The fatty acid has multiple functions including protecting the magnetic particle core from oxidation and/or hydrolysis in the presence of water, which can significantly reduce the magnetization of the nanoparticle (Hutten, et al. (2004) J. Biotech. 112:47-63); stabilizing the nanoparticle core; improving biocompatibility; and serving as an interface for anchoring the hydrophobic groups of the surfactant. The particular fatty acid selected can be dependent upon the magnetic particle core, the desired fluidity, the intended use (e.g., imaging or drug delivery), etc. The fatty acid can be saturated or unsaturated, and in particular embodiments, the fatty acid is unsaturated. Exemplary saturated fatty acids include lauric acid, myristic acid, palmitic acid, stearic acid, and arachidic acid. Exemplary unsaturated fatty acids include oleic acid, linoleic acid, linolenic acid, arachidonic acid and the like. The fatty acid can be synthetic or isolated from a natural source using established methods. Moreover, a fatty acid can be a derivative such as a fatty acid enol ester (i.e., a fatty acid reacted with the enolic form of acetone), a fatty ester (i.e., a fatty acid with the active hydrogen replaced by the alkyl group of a monohydric alcohol), a fatty amine or fatty amide, or in particular embodiments, a fatty alcohol. The fatty acid can be applied as a monolayer, wherein the thickness is engineered by controlling the chain length of the fatty acid. As such, the fatty acid component of the instant nanoparticle is generally 5 to 40% weight/weight with the magnetic particle core. As a total composition (i.e., magnetic particle core coated with a fatty acid and surfactant), the fatty acid is, in certain embodiments in the range of 10 to 30 weight % of the total composition. In other embodiments, the fatty acid is 15-25 weight % of the total composition. However, it is contemplated that higher percentages can be achieved when the fatty acid is applied as multiple layers.

Advantageously, the use of a surfactant in the instant nanoparticle compositions provides for increased circulation time in vivo. A surfactant, as used in the context of the instant invention is an organic compound that is amphipathic, i.e., containing both hydrophobic groups and hydrophilic groups. The hydrophobic groups of the surfactant anchor at the interface of the fatty acid corona and the hydrophilic groups extend into the aqueous phase, thereby conferring aqueous dispersity to the instant nanoparticle composition as well as increasing the hydrodynamic diameter of the instant composition upon hydration. Surfactants with a variety of chain lengths, hydrophilic-lipophilic balance (HLB) values and surfaces charges can be employed depending upon the application (e.g., the duration of time for which in vivo retention is desired). Surfactants with HLB values greater than 8 are particularly useful because of their high aqueous dispersity. In certain embodiments, the surfactant has an HLB value in the range of 8-18, so that the surfactant is anchored at the oleic acid-water interface. While PLURONIC® F-127 is exemplified herein, a PLURONIC® with a longer hydrophilic chain (e.g., PLURONIC® F-108) can be employed, as can TETRONIC® 908 and 1508 copolymers with polyethylene oxide (PEO) terminal blocks of molecular weight >5000 and polypropylene oxide (PPO) middle blocks of molecular weight >3000, di or tri block co-polymers such as PEG-PCL (polycaprolactone)-PEG, wherein HLB values >24. Such surfactants have been found to reduce adsorption plasma proteins on nanoparticles and significantly increase blood circulation half-life. Moreover, a surfactant can be a fatty acid esters (e.g. polyethyleneglycol distearate). Exemplary surfactants include, but are not limited to, PLURONIC® F-127, PLURONIC® F-108, PLURONIC® F-88, PLURONIC® F-68, TETRONIC® 908, TETRONIC® 1508, BRIJ® 92, TRITON® X-100, TRITON X®-405, Span20, HAMPOSYL®-O, TWEEN™-80, POLYSTEP® B-1 and POLYSTEP® F-9 and combinations thereof. In particular embodiments, the surfactant is a block co-polymer of ethylene oxide and propylene oxide. In other embodiments, the surfactant has a PEO:PPO:PEO composition of 70-265:30-70:70-265. In general, surfactants having longer hydrophilic chain lengths are particularly suitable, as longer hydrophilic chain lengths are associated with longer circulation times. For example, PLURONIC® with PEO-PPO-PEO block copolymers, such as PLURONIC® F-127 (PEO₁₀₀ PPO₆₅-PEO₁₀₀) and PLURONIC® F-68 (PEO₇₈PPO₃₀PEO₇₈), with PPO in the range of 30 to 60 and PEO in the range of 70 to 265 exhibit a long circulation time.

In certain embodiments, the nanoparticle composition of the instant invention has a magnetic particle core:fatty acid:surfactant ratio in the range of 3-4:1:4-5. Alternatively, a nanoparticle composition of the instant invention is, by weight, composed of 50-75% magnetic particle, 10-30% fatty acid and 10-30% surfactant. In still further embodiments, the instant nanoparticle has a polydispersity index in the range of ˜0.05 to ˜0.250 and a hydrodynamic diameter in the range of 180-200 nm. To achieve smaller diameters and suitable polydispersity indices, the nanoparticles can be dispersed in the aqueous phase by sonication, magnetic separation, or passed through a high-pressure homogenizer and extruder (e.g., supplied by AVESTIN® Inc., Ottawa, Canada) to remove larger particles and simultaneously sterilize the nanoparticle composition. Moreover, as exemplified herein, dispersion stability can be increased by the addition of PEG, which advantageously can also be used for conjugating targeting moieties to the instant nanoparticle composition.

Thus, one embodiment of the instant invention embraces a functional group. The functional group can be obtained by directly modifying the surfactant (e.g., prior to being coated on the fatty acid-stabilized magnetic nanoparticle) or by combining the surfactant (e.g., during the coating process) with a compound harboring a functional group (e.g., PEG; derivatives of PEG such as PEG terminated with succinimidyl glutarate, maleimide, succinimidyl succinate, tiol, amino, diacrylate, or acrylate; polycaprolactone terminated with amino, thiol, or hydroxyl groups; polyvinyl amines; polyvinyl alcohol; ethylene ethyl acrylate copolymer; maleic anhydride grafted polymer; epoxy polymers; graft copolymer consisting of polycarbonate (PC) as a main-chain and styrene-acrylonitrile copolymer (PSAN); polystyrene (PS) and modified PSAN as a branch polymer; vinyl co-polymers;, poly-L-lysine, and polyethylenimines (PEI)). A functional group is intended to include amine, hydroxyl, carboxyl, and aldehyde groups, as well as an amide group under suitable pH and buffer conditions. By way of illustration, a surfactant such as PLURONIC® can be modified with polyacrylic acid (PAA) by dispersion/emulsion polymerization to achieve carboxyl functional groups (Bromberg (1998) Ind. Eng. Chem. Res. 37:4267-4274).

As used herein, a targeting moiety is any molecule that can be conjugated to a functional group on a nanoparticle of the present invention to facilitate, enhance, or increase the transport of the nanoparticle to or into a target cell, tissue, or structure (e.g., a cancer cell, an immune cell, a pathogen, the brain, a blood clot, etc.). In particular embodiments, the targeting moiety is used in combination with an external magnetic field to facilitate targeting of the instant nanoparticle composition. Targeting moieties include polypeptides, peptides, antibodies, antibody fragments, oligonucleotide-based aptamers with recognition pockets, and small molecules that bind to specific cell surface receptors or polypeptides on the outer surface of the cell wherein the cell surface receptors or polypeptides are specific to that cell type. For example, a variety of protein transduction domains, including the HIV-1 Tat transcription factor, Drosophila Antennapedia transcription factor, as well as the herpes simplex virus VP22 protein have been shown to facilitate transport of proteins into the cell (Wadia and Dowdy (2002) Curr. Opin. Biotechnol. 13:52-56). Further, an arginine-rich peptide (Futaki (2002) Int. J. Pharm. 245:1-7), a polylysine peptide containing Tat PTD (Hashida, et al. (2004) Br. J. Cancer 90(6):1252-8), PTD-4 (Ho, et al. (2001) Cancer Res. 61:474-477), transportin (Schwartz and Zhang (2000) Curr. Opin. Mol. Ther. 2:2), Pep-1 (Deshayes, et al. (2004) Biochemistry 43(6):1449-57) or an HSP70 protein or fragment thereof (WO 00/31113) is suitable for targeting a nanoparticle of the present invention. Not to be bound by theory, it is believed that such transport domains are highly basic and appear to interact strongly with the plasma membrane and subsequently enter cells via endocytosis (Wadia, et al. (2004) Nat. Med. 10:310-315). Animal model studies indicate that chimeric proteins containing a protein transduction domain fused to a full-length protein or inhibitory peptide can protect against ischemic brain injury and neuronal apoptosis; attenuate hypertension; prevent acute inflammatory responses; and regulate long-term spatial memory responses (Blum and Dash (2004) Learn. Mem. 11:239-243; May, et al. (2000) Science 289:1550-1554; Rey, et al. (2001) Circ. Res. 89:408-414; Denicourt and Dowdy (2003) Trends Pharmacol. Sci. 24:216-218).

Suitable small molecule targeting moieties which can be conjugated to a nanoparticle of the present invention include, but are not limited to, nonpeptidic polyguanidylated dendritic structures (Chung, et al. (2004) Biopolymers 76(1):83-96) or poly[N-(2-hydroxypropyl)methacrylamide] (Christie, et al. (2004 ) Biomed. Sci. Instrum. 40:136-41).

Moreover, peptide hormones such as bombesin, stomatostatin and luteinizing hormone-releasing hormone (LHRH) or analogs thereof can be used as targeting moieties. Cell-surface receptors for peptide hormones have been shown to be overexpressed in tumor cells (Schally (1994) Anti-Cancer Drugs 5:115-130; Lamharzi, et al. (1998) Int. J. Oncol. 12:671-675) and the ligands to these receptors are known tumor cell targeting agents (Grundker, et al. (2002) Am. J. Obstet. Gynecol. 187(3):528-37; WO 97/19954). Carbohydrates such as dextran having branched galactose units (Ohya, et al. (2001) Biomacromolecules 2(3):927-33), lectins (Woodley (2000) J. Drug Target. 7(5):325-33), and neoglycoconjugates such as Fucalpha1-2Gal (Galanina, et al. (1998) Int. J. Cancer 76(1):136-40) may also be used as targeting moieties to treat, for example, colon cancer. It is further contemplated that an antibody or antibody fragment which binds to a protein or receptor, which is specific to a tumor cell, can be used to as a cell-surface targeting moiety. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. Exemplary antibody targeting moieties include an anti-HER-2 antibody (Yamanaka, et al. (1993) Hum. Pathol. 24:1127-34; Stancovski, et al. (1994) Cancer Treat Res. 71:161-191) for targeting breast cancer cells and bispecific monoclonal antibodies composed of an anti-histamine-succinyl-glycine Fab′ covalently coupled with an Fab′ of either an anticarcinoembryonic antigen or an anticolon-specific antigen-p antibody (Sharkey, et al. (2003) Cancer Res. 63(2):354-63).

Transferrin is another suitable targeting moiety which has been extensively investigated as a ligand for targeting of antineoplastic agents (Qian, et al. (2002) Pharmacol. Rev. 54:561-587; Widera, et al. (2003) Adv. Drug. Deliv. Rev. 55:1439-1466). Moreover, transferrin has been used to deliver therapeutic agents across the blood-brain barrier, which is otherwise impermeable to most therapeutic agents (Pardridge (2002) Adv. Exp. Med. Biol. 513:397-430; Bickel, et al. (2001) Adv. Drug Deliv. Rev. 46:247-279).

Standard methods employing homobifunctional or heterobifunctional crosslinking reagents such as carbodiimides, sulfo-NHS esters linkers, and the like can be used for conjugating or operably attaching the targeting moiety to a functional group of a nanoparticle of the present invention, as can aldehyde crosslinking reagents, such as glutaraldehyde. For example, conjugation to carboxyl groups generated on a modified surfactant (e.g., PEO-PPO-PEO-PAA) can be carried out using a coupling agent such as 1-ethyl-3-(3-dimethylaminopropyl)carbodiimide (EDC; Bromberg & Salvati (1999) Bioconjug. Chem. 10:678-86). Moreover, methods such as epoxy activation (Labhasetwar, et al. (1998) J. Pharm. Sci. 87:1229-34) can be employed for conjugation of targeting moieties to hydroxyl functional groups. Other suitable chemistries are well-known to the skilled artisan.

Nanoparticle compositions produced in accordance with the instant invention can be used in a variety of applications including, but not limited to, delivery of therapeutic agents for the prevention and treatment of diseases and conditions, magnetic nanoparticle-mediated thermotherapy (see, e.g., U.S. patent application Ser. No. 10/696,399), magnetic resonance imaging, delivery of detectable moieties for diagnostic imaging (e.g., PET, SPECT, optical), or combinations thereof.

Given that small paramagnetic or superparamagnetic particles of ferrite (iron oxide Fe₃O₄ or Fe₂O₃) are routinely used as paramagnetic contrast medium in magnetic resonance imaging (MRI), the instant nanoparticle composition can be directly employed in MRI. The instant magnetic nanoparticles are advantageously used over conventional contrast agents because the instant nanoparticles provide increased in vivo retention times and stability (i.e. , reduced oxidation and/or hydrolysis). For example, wherein convention iron-oxide-based contrast agents lose signal intensity at 1 and 2 days, it is contemplated that the improved uptake and stability of the instant nanoparticle composition will improve signal stability over time thereby facilitating MRI analysis. Thus, the instant nanoparticle composition can be injected into a subject in need of imaging and MRI analysis can be conducted according to standard methods. As disclosed herein, magnetic nanoparticles localize to damaged tissues in the presence and absence of an external magnetic field, albeit to a greater extent when an external magnetic field is applied. Accordingly, particular embodiments of the instant invention embrace exposing a subject in need of MRI imaging to an external magnetic field to facilitate imaging of a selected part of the body (e.g., the brain, a tumor, lesions, blood clot, etc.). Exposure to an external magnetic field can be achieved by, e.g., placing a magnet over the selected part of the body being targeted either before or just after administration of the nanoparticle composition.

While the nanoparticle composition of the instant invention can be used directly for diagnostic imaging, particular embodiments of the instant invention encompass intercalation or insertion of a detectable moiety or at least one therapeutic agent within the fatty acid corona of the nanoparticle for facilitating imaging of the detectable moiety or increasing the efficacy of the therapeutic agent.

A detectable moiety is a compound or molecule that is readily detectable either by its presence, or by its activity, which results in the generation of a detectable signal. Examples of detectable moieties include, but are not limited to, radioisotopes (e.g., primary positron-emitting radionuclides used in PET, radionuclides such as Technetium-99 m and Thallium-201 used in SPECT), fluorescent dyes (e.g., fluorescamine, coumarin, pyrene and its derivatives, rhodamine and its derivatives, and ALEXA® derivatives), infrared dyes, near infrared dyes (e.g., ALEXA FLUOR®, CY5.5™), chelators, fluorescent or luminescent proteins (e.g., GFP, luciferase, etc.), quantum dots, and nanocystals. A magnetic nanoparticle composition containing a detectable moiety can be injected into a subject in need of diagnostic imaging and imaging analysis can be conducted according to routine methods in the art of medical imaging. As with MRI imaging, use of the instant nanoparticle composition to delivery detectable moieties facilitates diagnostic imaging analysis by increasing uptake and retention of the detectable moiety. Moreover, imaging of a selected body part can be achieved by exposing a subject in need of diagnostic imaging to an external magnetic field.

In addition to diagnostic imaging, it is contemplated that localizing a magnetic nanoparticle containing a detectable moiety to a tumor can be used to facilitate identification and removal of tumor cells during surgery. Moreover, it is contemplated that image analysis can be used in combination with therapeutic treatment (e.g., chemotherapy) to monitor drug distribution and uptake, and tumor regression.

A therapeutic agent, in the context of the instant invention, encompasses any natural or synthetic, organic or inorganic molecule or mixture thereof for preventing or treating a disease or condition in a subject. As used herein, a therapeutic agent includes any compound or mixture of compounds which produces a beneficial or useful result. In certain embodiments of the invention, the nanoparticle composition contains at least two, three, four or more therapeutic agents. In other embodiments, the nanoparticle composition contains at least one therapeutic agent and at least one detectable moiety. In a still further embodiment, the nanoparticle composition contains a targeting moiety, at least one therapeutic agent, and at least one detectable moiety. Therapeutic agents are distinguishable from such components as vehicles, carriers, diluents, lubricants, binders and other formulating aids, and encapsulating, delivery or otherwise protective components. Examples of therapeutic agents include locally or systemically acting therapeutic agents which can be administered to a subject in need of treatment (i.e., exhibiting signs or symptoms associated with a particular disease or condition) according to standard methods of delivering nanoparticles (e.g., oral, topical, intralesional, injection, such as subcutaneous, intradermal, intratumoral, intramuscular, intraocular, or intra-articular injection, and the like) in the presence or absence of an external magnetic field. Examples of therapeutic agents for the prevention or treatment of diseases and conditions include, but are not limited to, anti-oxidants (e.g., superoxide dismutase, catalase, glutathione peroxidase, glutathione reductase, glutathione-S-transferase), anti-infectives (including antibiotics, antivirals, fungicides, scabicides or pediculicides), antiseptics (e.g., benzalkonium chloride, benzethonium chloride, chlorohexidine gluconate, mafenide acetate, methylbenzethonium chloride, nitrofurazone, nitromersol and the like), steroids (e.g., estrogens, progestins, androgens, adrenocorticoids, and the like), therapeutic polypeptides (e.g. insulin, erythropoietin, morphogenic proteins such as bone morphogenic protein, and the like), analgesics and anti-inflammatory agents (e.g., aspirin, ibuprofen, naproxen, ketorolac, COX-1 inhibitors, COX-2 inhibitors, and the like), cancer therapeutic agents (e.g., paclitaxel, mechliorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen, and the like), narcotics (e.g., morphine, meperidine, codeine, and the like), local anesthetics (e.g., the amide- or anilide-type local anesthetics such as bupivacaine, dibucaine, mepivacaine, procaine, lidocaine, tetracaine, and the like), antiangiogenic agents (e.g., combrestatin, contortrostatin, anti-VEGF, and the like), neuroprotective agents (e.g., neurotrophins such as BDNF), polysaccharides, vaccines, antigens, nucleic acids (e.g., DNA and other polynucleotides, antisense oligonucleotides, and the like), etc. As exemplified herein, the therapeutic agent can be added after the formulation of the nanoparticle or alternatively, can be inserted during formulation of the nanoparticle, e.g., with the fatty acid. Advantageously, use of the instant nanoparticle composition to deliver therapeutic agents can increase drug retention and targeting which results in improved drug efficacy so that lower amounts of therapeutic drug can be administered thereby reducing side effects and costs associated with treatment.

As will be appreciated by the skilled artisan, the nanoparticle compositions of the present invention can further contain additional pharmaceutically acceptable fillers, excipients, binders, etc. depending on, e.g., the route of administration and the therapeutic agents or detectable moieties used. A generally recognized compendium of such ingredients and methods for employing the same is Remington: The Science and Practice of Pharmacy, Alfonso R. Gennaro, editor, 20th ed. Lippincott Williams & Wilkins: Philadelphia, Pa., 2000.

The invention is described in greater detail by the following non-limiting examples.

### EXAMPLE 1

### Materials

Iron (III) chloride hexahydrate (FeCl₃.6H₂O) pure granulated, 99%, Iron (II) chloride tetrahydrate (FeCl₂.4H₂O) 99+%, ammonium hydroxide (5M), and oleic acid were purchased from Fisher Scientific (Pittsburgh, Pa.). PLURONIC® F-127 was from BASF Corporation (Mt. Olive, N.J.). TWEEN®-80 was obtained from Sigma-Aldrich (St. Louis, Mo.). Doxorubicin hydrochloride was from Dabur Research Foundation (Ghaziabad, India). De-ionized water purged with nitrogen gas was used in all the steps involved in the synthesis and formulation of magnetic nanoparticles.

### EXAMPLE 2

### Synthesis of Magnetic Nanoparticles

Aqueous solutions of 0.1 M Fe(III) (30 mL) and 0.1 M Fe(II) (15 mL) were mixed, and 3 mL of 5 M ammonia solution was added drop-wise over one minute while stirring on a magnetic stir plate. The stirring continued for 20 minutes under a nitrogen-gas atmosphere. The particles obtained were washed three times using ultracentrifugation (30,000 rpm for 20 minutes at 10° C.) with nitrogen-purged water. The iron-oxide nanoparticle yield, determined by weighing the lyophilized sample of the preparation, was 344 mg.

### EXAMPLE 3

### Formulations of Magnetic Nanoparticles

Formulations with different weight ratios of oleic acid to iron-oxide nanoparticles were prepared to optimize the amount of oleic acid required to completely coat iron-oxide nanoparticles. For this purpose, oleic acid was added (6-250 mg corresponding to 1.7 weight % to 41.0 weight % of the total formulation weight, i.e., iron-oxide nanoparticles plus oleic acid) to the above solution of Fe (III) and Fe (II) following the addition of ammonia solution. The formulations were heated to 80° C. while stirring for 30 minutes to evaporate the ammonia, and then cooled to room temperature. The black precipitate thus obtained was washed twice with 15 mL of water; the excess oleic acid formed an emulsion as apparent from the turbid nature of the supernatant. The precipitate was lyophilized for 2 days at −60° C. and 7 μm Hg vacuum (LYPHLOCK® 12; LABCONCO®, Kansas City, Mo.).

To study the effect of PLURONIC® on aqueous dispersity of oleic acid-coated iron-oxide nanoparticles, different amounts of PLURONIC® (25-500 mg corresponding to 5.6 weight % to 54.0 weight % of total formulation weight, i.e., iron-oxide nanoparticles plus oleic acid plus PLURONIC®) were added to the optimized composition of oleic acid-coated iron-oxide nanoparticles as determined above. PLURONIC® was added to the dispersion of oleic acid-coated nanoparticles (the dispersion was cooled to room temperature but not lyophilized) and stirred overnight in a closed container to minimize exposure to atmospheric oxygen thereby preventing oxidation of the iron-oxide nanoparticles. These particles were washed with water to remove soluble salts and excess PLURONIC®.

Particles were separated using two methods. In one method, particles were separated by ultracentrifugation at 30,000 rpm (OPTIMA® LE-80K; Beckman Coulter, Inc., Palo Alta, Calif.) using a fixed angle rotor (50.2 Ti) for 30 minutes at 10° C. The supernatant was discarded and the sediment was redispersed in 15 mL of water by sonication in a water-bath sonicator (FS-30, Fisher Scientific) for 10 minutes. The suspension was centrifuged as above and the sediment was washed three times with water. Nanoparticles were resuspended in water by sonication as above for 20 minutes and centrifuged at 1000 rpm for 20 minutes at 7-11° C. to remove any large aggregates. The supernatant containing oleic acid-PLURONIC®-stabilized nanoparticles was collected and used for drug loading.

In a second method, particles were separated by magnetic separation, which was carried out using two magnets (placed with opposite poles facing each other) on the parallel faces of the cuvette containing the particles. Particles recovered with magnetic separation were found to be more uniform in particle size as compared to those which were recovered using ultracentrifugation (polydispersity index=0.115 vs 0.262). Lower polydispersity index represents more uniform particle size distribution. There was no significant difference in the mean particle size.

### EXAMPLE 4

### Physical Characterization of Nanoparticles

Dynamic Laser Light Scattering and Zeta Potential Measurements. For measuring the particle size of oleic acid-coated nanoparticles, each sample was dispersed in hexane (0.1 mg/mL) using a water-bath sonicator for five minutes and particle size was measured using a glass cuvette (Zeta plus zeta potential analyzer, Brookhaven Instruments Corporation, Holtsville, N.Y.). An identical procedure was used for measuring the particle size of oleic acid-PLURONIC®-stabilized nanoparticles, except that the nanoparticle suspension was prepared in water (2 μg/mL) and the size was measured using a polystyrene cuvette (Brookhaven Instruments Corporation). The same suspension was diluted for measuring the Zeta potential of particles (Brookhaven Instruments Corporation).

Transmission Electron Microscopy (TEM). A drop of an aqueous dispersion of oleic acid-PLURONIC® stabilized nanoparticles was placed on a formvar-coated copper TEM grid (150 mesh; Ted Pella Inc., Redding, Calif.) and was allowed to air dry. Particles were imaged using a PHILIPS 201® transmission electron microscope (PHILIPS®/FEI Inc., Briarcliff Manor, N.Y.). The NIH ImageJ software was used to calculate the mean particle diameter from the TEM photomicrograph. Diameters of 50 particles were measured to calculate the mean particle diameter.

X-Ray Diffraction. The X-ray diffraction analysis of lyophilized samples of oleic acid-coated iron-oxide nanoparticles was carried out using a Rigaku D-Max/B horizontal diffractometer with Bragg-Brentano parafocusing geometry (Rigaku, The Woodlands, Tex.). The equipment uses a copper target X-ray tube with Cu Kα radiation. The parameters chosen for the measurement were: 2θ-steps of 0.02°, 6 seconds of counting time per step, and 2θ range from 20° to 80°. Approximately 15 mg of lyophilized sample was sprinkled onto a low-background quartz X-ray diffraction holder coated with a thin layer of silicone grease to retain the sample.

Thermogravimetric Analysis. Lyophilized samples (˜2 mg) of nanoparticles (oleic acid- and oleic acid-PLURONIC®-coated) were placed in aluminum sample cells (Fisher Scientific) and a thermogram for each sample was obtained using a Shimadzu thermogravimetric analyzer (TGA50; Shimadzu Scientific Instruments Inc., Columbia, Md.). Samples were heated at the rate of 15° C./minute under the flow of nitrogen gas set at an outlet pressure of 6-10 Kg/cm².

Fourier Transform Infrared (FT-IR) Spectroscopy. Measurements were carried out on a Nicolet AVATAR® 360 FT-IR spectrometer (Thermo Nicolet Corp., Madison, Wis.), and each spectrum was obtained by averaging 32 interferograms with resolution of 2 cm⁻¹. Pellets for FT-IR analysis were prepared by mixing the lyophilized samples of iron-oxide nanoparticle formulations with spectroscopic KBr powder.

Magnetization Studies. Magnetic measurements were carried out using a Quantum Design MPMS® SQUID magnetometer, and room-temperature measurements were performed using a MICROMAG™ 2900 alternating gradient field magnetometer (AGFM; PRINCETON MEASUREMENTS CORP.™, Princeton, N.J.). Zero-field-cooled (ZFC) and field-cooled (FC) magnetization measurements as functions of temperature were performed. For the ZFC measurement, each sample was cooled from 300 K to 10 K in zero field and the magnetization was measured as a function of temperature at 100 Oe as the sample was warmed. For the FC measurement, the sample was cooled in the measuring field and the magnetization was measured as the sample was cooled. Magnetization measurements as a function of field M(H) were performed at 10 K and 300 K. At 10 K, the saturation magnetization M_{S} and the coercive field H_{c} were determined by fitting the magnetization curve with an analytical ferromagnetic model and a diamagnetic contribution (χ) due to the background (Stearns & Cheng (1994) J. Appl. Phys. 75:6894-6899; Noyau, et al. (1988) IEEE Trans. Magn. 24:2494-2496).

$$M ( H ) = \frac{2}{π} {M}_{s} ArcTan ( ( \frac{H}{{H}_{c}} \pm 1 ) tan ( \frac{π s}{2} ) ) + χ H :$$

At 300 K, the M(H) loops were fit to a Langevin function weighted by a log-normal distribution of particle sizes.

### EXAMPLE 5

### Incorporation of Functional Groups

To a 20 mL solution of PEG (molecular weight 5000) in water was added 100 mg of oleic acid-coated iron-oxide nanoparticles to achieve nanoparticle:PEG ratios (weight:weight) of 1:1 and 1:10. The mixture was stirred on a magnetic stir-plate for 2 hours and 24 mg of PLURONIC® was subsequently added. The suspension was stirred overnight in a closed container, excess PLURONIC® and PEG were removed by overnight dialysis against water (SPECTROPORE®, molecular weight cut off of 100 KDa), and the suspension was lyophilized.

Conjugation to Targeting Moiety. Prior to incorporation into nanoparticles, PEG is conjugated to a targeting moiety, e.g., an antibody, using a condensation method. In a typical reaction, 3.2 mL of 2 M hexamethylene-diamine (HMD) is added to 1.0 mL of antibody solution (8.3 mg/mL in 0.1 M PBS, pH 7.4) and the pH is adjusted to 7.4. After mixing, 44 mg of fresh EDC is added to the mixture and the pH is readjusted to 6.8. The mixture is gently stirred on a magnetic stir plate for 3 hours at room temperature. The reaction is stopped by the addition of 1.0 mL of 1 M glycine, followed by incubation for 30 minutes at room temperature. Antibody-conjugated PEG is recovered by dialysis and incorporated with the surfactant coating as disclosed herein. The final nanoparticle composition is characterized for composition and structure by ¹H-NMR, ¹³C-NMR, FT-IR spectroscopy, fluorescamine detection of free amino groups.

### EXAMPLE 6

### Drug Loading in Magnetic Nanoparticles

Doxorubicin Loading. For incorporation in nanoparticles, hydrochloride salt of the drug (DOX.HCl) was converted to water-insoluble base (DOX) using established methods (Yolles, et al. (1978) Acta Pharm. Suec. 15:382-388). A methanolic solution of DOX (600 μL, 5 mg/mL) was added drop-wise while stirring to an aqueous dispersion of oleic acid-PLURONIC®-stabilized iron-oxide nanoparticles (30 mg of particles in 7 mL water). Stirring was continued overnight (˜16 hours) to allow partitioning of the drug into the oleic acid shell surrounding iron-oxide nanoparticles. Drug-loaded nanoparticles were separated from the unentrapped drug using a magnet (12200 Gauss; Edmund Scientific, Tonawanda, N.Y.). Nanoparticles were washed twice by re-suspending in distilled water and separated using a magnetic field.

To determine drug loading, a 200 μL aliquot of nanoparticle suspension was lyophilized and the weight of the lyophilized sample was measured. For drug extraction, 2 mL of 12.5% volume/volume methanolic solution in chloroform was added to the dried sample. The samples were shaken for 24 hours (Environ shaker, model no. 3527; Lab-Line Instruments, Melrose Park, Ill.). Since DOX has greater solubility in this combination of solvents than in methanol or chloroform alone, it was used for the extraction. Nanoparticles were centrifuged for 10 minutes at 16,000 g using an EPPENDORF® microcentrifuge (5417R; Eppendorf-Netheler-Hinz-GmbH, Hamburg, Germany). An aliquot (100 μL) of the supernatant was diluted to 1 mL with a methanol-chloroform mixture and the drug concentration was determined using a fluorescence spectrophotometer (Cary Eclipse; VARIAN® Inc., Walnut Creek, Calif.) at λₑₓ=485 nm and λₑₘ=591 nm. A standard plot was prepared under identical conditions to calculate the amount of drug loaded in the nanoparticles. There was no further increase in the amount of drug extracted when nanoparticles were kept for extraction for more than 24 hours.

Paclitaxel Loadings. To a 5 mg formulation of oleic acid-PLURONIC®-stabilized magnetic nanoparticles in 2 mL water, 100 μL of ethanolic solution of paclitaxel (5 mg/mL) was added and the suspension was stirred for 6 hours in a closed capped vial. The cap was removed and ethanol was allowed to evaporate overnight. Magnetite nanoparticles were separated from the free drug using a magnetic field and particles were washed two times with distilled water.

Paclitaxel and Doxorubicin Loading. As with single drug loading described above, an ethanolic solution of paclitaxel and doxorubicin were premixed while keeping the total drug concentration the same (5 mg/mL). The initial formulation contained 1:1 weight/weight ratio of paclitaxel and doxorubicin. Radioactive paclitaxel was used to analyze paclitaxel loading in magnetic nanoparticles whereas doxorubicin was determined by using a fluorescence spectrophotometer (λₑₓ=485 nm, λₑₘ=591 nm).

### EXAMPLE 7

### Kinetics of DOX Release

DOX-loaded nanoparticles were suspended in phosphate-buffered saline (154 mM, pH=7.4) containing 0.1% weight/volume TWEEN®-80, (PBS-TWEEN®-80). The release study was carried out in double diffusion cells, with the donor chamber filled with 2.5 mL of nanoparticle suspension (2 mg/mL) and the receiver chamber with 2.5 mL PBS-TWEEN®-80. The chambers were separated by a PVDF membrane of 0.1 μm porosity (DURAPORE®, VVLP; MILLIPORE® Corp., Billerica, Mass.). Nanoparticles do not cross the membrane but drug can diffuse freely. This was confirmed by analyzing the receiver chamber samples for iron content using a 220FS Flame Atomic Absorption Spectroscopy (VARIAN® Inc., Walnut Creek, Calif.). Cells were left on a shaker rotating at 110 rpm at 37° C. (Environ shaker), and buffer from the receiver chambers was completely withdrawn at different time intervals and replaced with fresh buffer. TWEEN®-80 was used in the buffer to maintain sink conditions during the release study. The samples were lyophilized and extracted with 12.5 volume % methanol in chloroform. DOX levels in the extracted samples were analyzed by measuring the fluorescence intensity at λₑₓ=485 nm and λₑₘ=591 nm. A standard plot for DOX was prepared under identical conditions, i.e., dissolving drug in TWEEN®-80 solution, lyophilizing the samples, and extracting the drug as described herein.

### EXAMPLE 8

### Cell Culture

PC3 (prostate cancer) and MCF-7 (breast cancer) cells purchased from American Type Culture Collection (ATCC, Manassas, Va.) were grown in RPMI 1640 medium supplemented with 10% fetal bovine serum and 100 μg/mL penicillin G and 100 μg/mL streptomycin (GIBCO BRL®, Grand Island, N.Y.) at 37° C. in a humidified and 5% CO₂ atmosphere.

### EXAMPLE 9

### Mitogenic Assay

PC3 and MCF-7 cells were seeded at 3,000 per well in 96-well plates (MICROTEST™; Becton Dickinson Labware, Franklin Lakes, N.J.) 24 hours prior to the experiment. Different concentrations of DOX (0.1 μM to 100 μM), either loaded in nanoparticles or as solutions, were added. For studies with DOX as a solution, a stock solution of hydrochloride salt (590 μg/mL) in 77% ethanol was prepared and 50 μL of this solution was diluted to 9 mL with medium containing serum to prepare a drug solution of 100 μM concentration. The maximum amount of alcohol used did not exceed 0.4 volume %, which does not affect cell growth. Drug solutions of lower concentrations were prepared by appropriate dilution of the above drug solution with serum-containing medium. A stock dispersion of drug-loaded iron-oxide nanoparticles was prepared in serum-containing medium so that the drug concentration was 100 μM. Nanoparticles without drug and medium were used as controls. Medium in the wells was replaced either with drug in solution or a dispersion of drug-loaded nanoparticles as described above. The medium was changed at 2 and 4 days following drug treatment, but no further dose of the drug was added. Cell viability was determined at 5 days post-treatment using a standard MTS assay (CELLTITER 96® AQᵤₑₒᵤₛ; PROMEGA®, Madison, Wis.). To each well was added 20 mL reagent, the plates were incubated for 75 minutes at 37° C. in the cell culture incubator, and color intensity was measured at 490 nm using a plate reader (BT 2000 Microkinetics Reader; BioTek Instruments, Inc., Winooski, Vt.). The effect of drug on cell proliferation was calculated as the percentage inhibition in cell growth with respect to the respective controls.

### EXAMPLE 10

### Confocal Laser Scanning Microscopy

MCF-7 cells were seeded in Bioptechs plates (Bioptechs, Butler, Pa.) at 50,000 cells/plate in 1 mL serum-containing medium 24 hours prior to the experiment. A dispersion of drug-loaded or void nanoparticles and drug solution (10 μM) were prepared in cell-culture medium as described herein. Cells were incubated either with drug in solution or a dispersion of drug-loaded nanoparticles for 2 hours, 24 hours or 48 hours. Cells were washed three times with PBS before imaging them under a confocal microscope (Zeiss Confocal microscope LSM410 equipped with argon-krypton laser; Zeiss, Thornwood, N.Y.) at λₑₓ=488 nm and a long-pass filter with a cut-on filter of 515 nm for detecting the emission light.

### EXAMPLE 11

### Statistical Analysis

Statistical analyses were performed using a Student's t-test. The differences were considered significant for p values of <0.05.

### EXAMPLE 12

### Uptake of Magnetic Nanoparticle in Rat Cerebral Ischemia Model

Ischemia was created by occlusion of the middle cerebral artery for one hour. A 550 μL suspension of magnetic nanoparticles (8 mg Fe/mL in PBS) was injected into rats (376-399 g) at a rate of 200 μL/minute using an infusion pump through the carotid artery. In the control, 550 μL PBS was injected. In one animal, magnetic field was created by placing a magnet on the brain (NdFeB Magnet, Magnetic field strength=12200 Gauss) prior to injecting a suspension of magnetic nanoparticles. After 1 hour, animals were perfused with PBS to wash off the blood. Brains were removed and left in perfluoroalkylether liquid (KRYTOX®, performance lubricant; DUPONT® de Nemours Inc., Wilmington, Del.) until subject to MRI analysis.

### EXAMPLE 13

### Assessment of Liver Function

Oleic acid-PLURONIC®-stabilized magnetic nanoparticle formulation (10 mg Fe/Kg in 500 μL PBS) was injected into rats (˜400 g) via tail vein. Blood was collected before and at a regular interval of time following injection. The collected blood was allowed to clot at room temperature, and centrifuged at about 3000 rpm for 10 minutes to separate serum. Serum samples were analyzed for various enzymes including aspartate aminotranserase, alanine aminotransferase, alkaline phosphatase, and gamma-glutamyl transferase to assess liver function.

Rats were euthanized 21 days post injection, blood was collected and serum was analyzed for iron levels and total iron binding capacity (TIBC). TIBC is an indirect measure of transferrin content which is produced in the liver and is indicative of liver function. A portion of liver was collected, fixed in the buffered formalin-saline at 4° C., and embedded in paraffin. Sections of 5 μm thickness were stained with Hematoxylin & Eosin.

### EXAMPLE 14

### Inert Gas Condensation of Fluids (IGC-F)

IGC-F is a physical vapor deposition technique that forms nanoparticles and deposits them directly into a surfactant-loaded fluid. A sputtering gun is used to produce an atomic or molecular vapor in a pressure of ˜0.1 torr of inert gas (e.g., Ar, He, or a combination thereof). The vapor atoms collide with the inert-gas molecules and form nanoclusters with a very narrow size distribution. The nanoclusters are deposited onto a rotating drum coated with a thin layer of surfactant-loaded fluid. As the drum rotates, the clusters are deposited into a reservoir.

The advantages of this technique are the narrow size distribution of the nanoparticles, the ability to vary the mean nanoparticle size from 5-50 nm, the flexibility to deposit any material that can be sputtered, including alloys, selection of a surfactant independent of the cluster fabrication process (so that nanoparticle size and surfactant are not correlated), and the ability to use reactive sputtering to create oxides, nitrides and carbides.

Coating of the IGC-F nanoparticles is achieved by extracting the nanoparticles from the deposition fluid using surfactant exchange or the deposition fluid can be used as part of the synthesis process.

What is claimed is:

1. A magnetic nanoparticle composition comprising a magnetic particle core coated with a fatty acid and surfactant.

2. The magnetic nanoparticle composition of claim 1 further comprising a functional group.

3. The magnetic nanoparticle composition of claim 1, further comprising at least one therapeutic agent.

4. The magnetic nanoparticle composition of claim 1, further comprising a detectable moiety.

5. A method for preparing a magnetic nanoparticle composition comprising coating a magnetic particle with a fatty acid and a surfactant.

6. The method of claim 5, further comprising incorporating a functional group, a therapeutic agent or detectable moiety.

7. A method for increasing the efficacy of a therapeutic agent comprising administering a composition of claim 3 to a subject in need of treatment with the therapeutic agent, thereby increasing the efficacy of the therapeutic agent in the subject.

8. The method of claim 7, wherein the magnetic nanoparticle composition is delivered to a selected part of the body by exposing the selected part of the body to an external magnetic field.

9. A method for facilitating magnetic resonance imaging comprising administering to a subject a composition of claim 1 thereby facilitating magnetic resonance imaging of the subject.

10. The method of claim 9, wherein the magnetic nanoparticle composition is delivered to a selected part of the body by exposing the selected part of the body to an external magnetic field.

11. A method for facilitating diagnostic imaging comprising administering to a subject a composition of claim 4 thereby facilitating diagnostic imaging of the subject.

12. The method of claim 11, wherein the magnetic nanoparticle composition is delivered to a selected part of the body by exposing the selected part of the body to an external magnetic field.
